(19) United States
(12) Patent Application Publication
(10) Pub. No.: US 2025/0001025 A1
(43) Pub. Date: Jan. 2, 2025

(54) OPTOELECTRONIC SEMICONDUCTOR CHIP AND DISINFECTION DEVICE

(71) Applicant: ams-OSRAM International GmbH, Regensburg (DE)

(72) Inventors: Laura KREINER, Regensburg (DE); Hans-Juergen LUGAUER, Sinzing (DE)

(21) Appl. No.: 18/708,210

(22) PCT Filed: Nov. 8, 2022
(86) PCT No.: PCT/EP2022/081111

§ 371 Date: May 8, 2024

(30) Foreign Application Priority Data
Nov. 9, 2021 (DE) 10 2021 129 106.2

(51) Int. Cl. A61L 2/10; H01L 33/20; H01L 33/46

(52) U.S. Cl. CPC A61L 2/10; H01L 33/20; H01L 33/46; A61L 2202/11

(57) ABSTRACT

The present disclosure provides an optoelectronic semiconductor chip including a semiconductor layer sequence with an active layer for generating primary radiation and an angle-selective filter on a first side of the semiconductor layer sequence. During operation, the semiconductor chip emits radiation in the UV range. The angle-selective filter is configured to let pass only radiation that hits the filter in a predefined angular range.

An optoelectronic semiconductor chip is specified. Moreover, a disinfection device is specified.

One task to be solved is to provide an improved optoelectronic semiconductor chip, for example with a predetermined radiation characteristic. The semiconductor chip is particularly suitable for use in a disinfection device, for example. Another task to be solved is to specify a disinfection device with such a semiconductor chip.

These tasks are solved, inter alia, by the subject-matter of independent claim 1 and claim 14. Advantageous embodiments and further developments are the subject of the dependent claims and are further apparent from the following description and the figures.

First, the optoelectronic semiconductor chip is specified.

According to at least one embodiment, the optoelectronic semiconductor chip comprises a semiconductor layer sequence with an active layer for generating primary radiation. The primary radiation is generated, for example, by recombination of holes and electrons in the active layer.

The semiconductor layer sequence is based, for example, on a III-V compound semiconductor material. The semiconductor material is, for example, a nitride compound semiconductor material, such as AlₙIn₁₋ₙ₋ₘGaₘN, or a phosphide compound semiconductor material, such as AlₙIn₁₋ₙ₋ₘGaₘP, or an arsenide compound semiconductor material, such as AlₙIn₁₋ₙ₋ₘGaₘAs or AlₙIn₁₋ₙ₋ₘGaₘAsP, where 0≤n≤1, 0≤m≤1 and m+n≤1. The semiconductor layer sequence may comprise dopants and additional components. For the sake of simplicity, however, only the essential components of the crystal lattice of the semiconductor layer sequence, i.e. Al, As, Ga, In, N or P, are specified, even if these may be partially replaced and/or supplemented by small amounts of other substances. For example, the semiconductor layer sequence is based on AlGaN.

The active layer of the semiconductor layer sequence includes, in particular, at least one pn junction and/or at least one quantum well structure in the form of a single quantum well, SQW for short, or in the form of a multi-quantum well structure, MQW for short. Preferably, the semiconductor chip comprises one, in particular exactly one, contiguous, in particular simply connected, active layer. Alternatively, the active layer can also be segmented.

The primary radiation is, for example, UV radiation, in particular UV-C or UV-B radiation.

A semiconductor chip is understood here and in the following as an element that can be handled separately and can be contacted electrically. A semiconductor chip is created in particular by singulation from a wafer composite. For example, the side surfaces of such a semiconductor chip comprise traces from the separation process of the wafer composite. A side surface of the semiconductor layer sequence is a surface of the semiconductor layer sequence delimiting the semiconductor layer sequence in the lateral direction.

A semiconductor chip preferably comprises exactly one originally contiguous area of the semiconductor layer sequence grown in the wafer composite. The semiconductor layer sequence of the semiconductor chip is preferably designed continuously. The lateral expansion of the semiconductor chip, measured parallel to the main extension plane of the active layer, is, for example, at most 1% or at most 5% or at most 10% greater than the lateral expansion of the active layer or the semiconductor layer sequence. This means that the lateral dimensions of the semiconductor chip are essentially defined by the lateral dimensions of the semiconductor layer sequence or the active layer. Lateral expansion is understood here and in the following in particular as an extension or expansion in any lateral direction. A lateral direction is a direction parallel to the main extension plane of the active layer.

The semiconductor chip may still comprise the growth substrate on which the semiconductor layer sequence was grown. For example, the semiconductor chip is a flip chip. Alternatively, the semiconductor chip can be a surface emitter, in particular a so-called thin film chip. In this case, the growth substrate is removed, for example.

According to at least one embodiment, the semiconductor chip comprises an angle-selective filter on a first side of the semiconductor layer sequence. The first side is in particular a side of the semiconductor layer sequence delimiting the semiconductor layer sequence, for example in the direction perpendicular to the main extension plane of the active layer. The first side can be a main side of the semiconductor layer sequence whose lateral expansion in any direction is, for example, greater than the thickness of the semiconductor layer sequence, measured perpendicular to the main extension plane of the active layer. The angle-selective filter can cover most of the first side of the semiconductor layer sequence, for example at least 80% or at least 90%.

The angle-selective filter can be applied directly to the first side of the semiconductor layer sequence or spaced from the first side by an intermediate layer. Preferably, the angle-selective filter is arranged close to the active layer, for example at a distance from the active layer that is at most as great as the thickness or half the thickness of the semiconductor layer sequence. A radiation decoupling surface of the semiconductor chip, via which a large part, for example at least 90%, of the radiation emitted by the semiconductor chip is decoupled during operation, is located, for example, on a side of the angle-selective filter opposite the semiconductor layer sequence. In other words, the angle-selective filter can be arranged between the semiconductor layer sequence and the radiation decoupling surface. The radiation decoupling surface can be partially or completely formed by the angle-selective filter.

The angle-selective filter is preferably already applied to the semiconductor layer sequence during wafer bonding and singulated during singulation into individual semiconductor chips. For example, side surfaces of the filter comprise traces of the separation process. A lateral expansion of the filter, for example, essentially corresponds to the lateral expansion of the semiconductor layer sequence, for example with a deviation of at most 10% or at most 5%. One or more side surfaces of the angle-selective filter can be flush with side surfaces of the semiconductor layer sequence.

According to at least one embodiment, the semiconductor chip emits radiation in the UV range during operation. The emitted radiation may be UV-B or UV-C radiation, for example. For example, the radiation emitted by the semiconductor chip is largely or completely formed by the primary radiation. This means, for example, that the semiconductor chip is free of a conversion element. Alternatively, however, the primary radiation can also be partially or completely converted into UV radiation by a conversion element of the semiconductor chip, which then leaves the semiconductor chip. In this case, the conversion element is arranged between the filter and the semiconductor layer sequence, for example.

According to at least one embodiment, the angle-selective filter is configured to let pass only radiation, in particular only UV radiation and/or primary radiation, which hits the filter in a predefined angular range. The predefined angular range is, for example, an angular range between 0° and α, where an angle of 0° is the direction parallel to a normal to the filter or to the main extension plane of the semiconductor layer sequence. The 0° direction can be the main emission direction of the semiconductor chip, i.e. the direction along which the greatest radiation intensity is emitted.

After passing through the angle-selective filter, the radiation leaves the semiconductor chip, preferably without further refraction and/or scattering and/or conversion. In particular, the radiation leaving the semiconductor chip comprises predominantly radiation in the predefined angular range. For example, at least 90% or at least 95% of the radiation emitted by the semiconductor chip is emitted in the predefined angular range.

In at least one embodiment, the optoelectronic semiconductor chip comprises a semiconductor layer sequence with an active layer for generating primary radiation and an angle-selective filter on a first side of the semiconductor layer sequence. During operation, the semiconductor chip emits radiation in the UV range. The angle-selective filter is configured to let pass only radiation that hits the filter in a predefined angular range.

The present invention is based, inter alia, on the realization that a narrow emission characteristic is advantageous for many UV radiation applications. For example, generated UV radiation for disinfection applications can be used much more efficiently if the absorption in the side surfaces of a housing of the disinfection device is reduced. With comparable requirements for white light, a component with a lens is typically used in this case to focus the emitted light into the desired angular cone. However, there are few suitable materials for applications in the UV range and these are either very expensive or difficult to process.

Instead of shaping the radiation characteristics at component level via lenses, in the present invention the emitted light is directed directly at chip level through the angle-selective filter into the corresponding angular range. As a result, high efficiency can be achieved in the application even without a lens.

According to at least one embodiment, the filter is a dielectric filter. In particular, the filter may comprise or consist of one or more dielectric materials.

According to at least one embodiment, the filter comprises a plurality of dielectric layers. For example, layers with a higher and lower refractive index are alternately stacked on top of each other. The refractive index refers in particular to the UV radiation and/or primary radiation emitted by the semiconductor chip, for example to the wavelength at which the UV radiation and/or primary radiation has a maximum intensity.

The dielectric filter may comprise at least four or at least ten or at least 50 dielectric layers, for example. Main extension planes of the dielectric layers each run parallel to the main extension plane of the active layer, for example. The dielectric layers are stacked on top of each other, for example in a direction away from the semiconductor layer sequence and/or in a direction perpendicular to the main extension plane of the active layer.

According to at least one embodiment, the filter comprises at least one layer comprising or consisting of HfO and/or at least one layer comprising or consisting of SiO₂.

According to at least one embodiment, the semiconductor chip comprises a deflection structure for deflecting the radiation generated in the semiconductor chip. In particular, the deflection structure is configured to change the angular distribution of the radiation hitting the deflection structure so that as much as possible of the radiation generated in the semiconductor chip hits the angle-selective filter at some point in the predefined angular range.

According to at least one embodiment, the deflection structure comprises a structuring, for example a roughening, of the semiconductor layer sequence. For example, the first side of the semiconductor layer sequence is specifically structured or roughened. This can be achieved by an etching or grinding process. A root square roughness (root mean square roughness) of the structured side of the semiconductor layer sequence is, for example, at least 10 nm or at least 50 nm or at least 100 nm. Alternatively or additionally, the roughness can be at most 2 μm or at most 1 μm or at most 500 nm.

A planarization layer can be applied to the structuring of the semiconductor layer sequence, which is essentially smooth on a side facing away from the structuring, for example with a roughness of at most 5 nm. The planarization layer can be made of a material that is transparent to UV radiation and/or the primary radiation and/or of a material with a lower refractive index than the semiconductor material for the corresponding radiation. For example, the planarization layer comprises or consists of SiO₂ or Al₂O₃.

According to at least one embodiment, the deflection structure comprises a beveled mesa edge of the semiconductor layer sequence. The mesa edge extends, for example, transversely to the main extension plane of the active layer or to the first side of the semiconductor layer sequence. For example, the mesa edge forms an angle of at least 10° or at least 20° or at least 30° and/or of at most 80° or at most 70° or at most 60° with the main extension plane or the first side. The active layer can be adjoin the mesa edge or a part of the mesa edge can be formed by the active layer. The mesa edge preferably extends from the first side of the semiconductor layer sequence to a second side of the semiconductor layer sequence opposite the first side, the second side also delimiting the semiconductor layer sequence. In other words, the mesa edge may extend over the entire thickness of the semiconductor layer sequence. The mesa edge can be flat within the manufacturing tolerances. In particular, the mesa edge forms a side surface of the semiconductor layer sequence.

The semiconductor chip may comprise a plurality of such beveled mesa edges. For example, several or all side surfaces of the semiconductor layer sequence are each formed by at least one beveled mesa edge.

According to at least one embodiment, the semiconductor chip comprises a mirror coating on one side of the semiconductor layer sequence. The mirror coating can be configured to reflect the radiation generated in the semiconductor chip, in particular UV radiation and/or primary radiation. For example, the mirror coating comprises a reflectance for the radiation of at least 80% or at least 90% or at least 95%.

The mirror coating may, for example, be arranged on one or more or all side surfaces of the semiconductor layer sequence and/or on a main side, for example the first or second side, of the semiconductor layer sequence. The mirror coating can cover most of the side of the semiconductor layer sequence covered by it, for example at least 75% or at least 90% or completely.

The mirror coating may comprise or consist of a metal such as Ag, Al or Au, for example. The mirror coating can be arranged on the beveled mesa edge of the semiconductor layer sequence. The mirror coating can be arranged on the structured side of the semiconductor layer sequence or on a side opposite to it.

According to at least one embodiment, the primary radiation and/or the radiation emitted by the semiconductor chip is radiation with an intensity maximum, in particular a global intensity maximum, at 320 nm or less, or a global intensity maximum at 280 nm or less. The maximum intensity can be at least 80 nm or at least 100 nm.

According to at least one embodiment, the semiconductor chip comprises a growth substrate of the semiconductor layer sequence. For example, the growth substrate comprises or consists of sapphire. The growth substrate is preferably not thinned, i.e. comprises the same thickness as before the singulation from the wafer composite. The growth substrate can then be the carrier stabilizing the semiconductor layer sequence, in particular the only carrier stabilizing the semiconductor layer sequence, of the semiconductor chip. In this case, the semiconductor chip can be a flip chip. The growth substrate is then arranged between the filter and the semiconductor layer sequence, for example. Contact elements, in particular both the cathode and the anode, can be arranged on a side of the semiconductor layer sequence facing away from the growth substrate.

According to at least one embodiment, the deflection structure comprises a structuring of one side of the growth substrate, for example the side of the growth substrate facing the semiconductor layer sequence. The structuring may be a nanostructure. This means that the growth substrate can be a so-called nano-patterned sapphire substrate (nano-PSS). The structures of the structuring of the growth substrate can, for example, comprise structure sizes in the range between 50 nm and 400 nm inclusive, for example between 100 nm and 300 nm inclusive.

Such structure sizes allow good growth on the growth substrate for a semiconductor layer sequence based on a nitride compound semiconductor material. For example, the first grown layer of the semiconductor layer sequence is then an AlN layer.

According to at least one embodiment, at least one side surface, preferably several or all side surfaces, of the growth substrate is mirror-coated. All the features disclosed in connection with the mirror coating of one side of the semiconductor layer sequence described above also apply to the mirror coating of the sides of the growth substrate. A side surface of the growth substrate is a surface of the growth substrate delimiting the growth substrate in the lateral direction. The side surfaces of the growth substrate can be flush with the side surfaces of the semiconductor layer sequence. Side surfaces of the growth substrate may also comprise traces of the separation process.

According to at least one embodiment, the semiconductor chip is a thin film chip. In this case, the growth substrate is removed or thinned. In this case, a carrier stabilizing the semiconductor layer sequence or the semiconductor chip is different from the growth substrate. The carrier may comprise or consist of organic material, for example plastic. The semiconductor layer sequence is then arranged between the carrier and the filter, for example.

The thin-film chip can comprise a housing body that surrounds the semiconductor layer sequence in a frame-like manner in the lateral direction. The housing body can also form the carrier. The thin-film chip can be a so-called chip-size package. The housing body may comprise or consist of plastic.

According to at least one embodiment, the predefined angular range comprises angles of at most 30° or at most 20° with respect to the main emission direction and/or the 0° direction. Preferably, the predefined angular range comprises all angles from 0° to 30° or from 0° to 20°. UV radiation and/or primary radiation that hits the angle-selective filter at angles greater than the maximum angle, for example 30° or 20°, does not pass through the filter but is reflected back into the semiconductor layer sequence.

The semiconductor chip described herein can be used in particular for disinfection. For example, the semiconductor chip can be used in a disinfection apparatus or a disinfection device.

Next, the disinfection device is specified.

According to at least one embodiment, the disinfection device comprises a housing with a receiving region for receiving an object to be disinfected. The housing comprises, for example, an interior space which is surrounded by a wall of the housing. The receiving region can be formed by the interior space or can be arranged in the interior space.

According to at least one embodiment, the disinfection device comprises an optoelectronic semiconductor chip according to at least one of the embodiments described herein. Accordingly, all features disclosed in connection with the semiconductor chip are also disclosed for the disinfection device and vice versa.

According to at least one embodiment, the semiconductor chip is arranged with respect to the housing in such a way that radiation emitted by the semiconductor chip during operation is emitted onto the receiving region. The semiconductor chip may, for example, be attached to the housing and/or integrated into the housing. The disinfection device may be a portable disinfection device.

The disinfection device can, for example, be configured to disinfect liquids, gases and/or solids, for example medical devices. In this case, the receiving region can be configured to receive a liquid or a gas or to allow a liquid or a gas to pass through or to receive a solid.

In the following, an optoelectronic semiconductor chip described herein and a disinfection device described herein are explained in more detail with reference to drawings on the basis of exemplary embodiments. Identical reference signs indicate identical elements in the individual figures. However, no references to scale are shown; rather, individual elements may be shown in exaggerated size for better understanding. Insofar as elements in the various figures correspond in their function, their description is not repeated for each of the following figures. For reasons of clarity, elements may not be provided with corresponding reference signs in all figures.

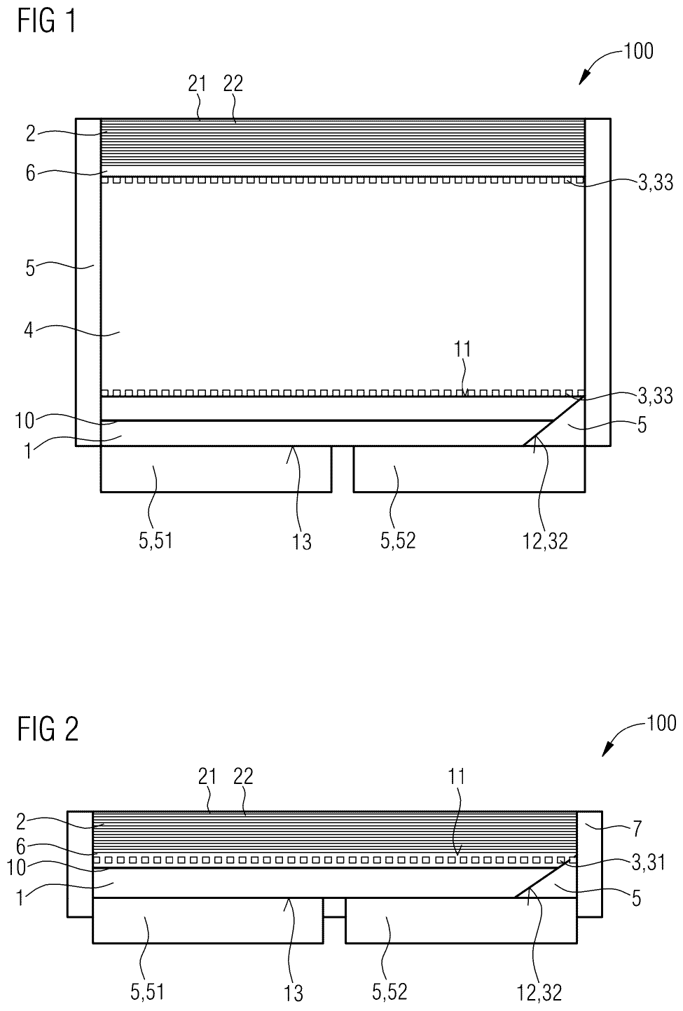

FIGS. 1 to 3 show various exemplary embodiments of an optoelectronic semiconductor chip, each in cross-sectional view,

FIG. 4 shows a modification of a disinfection device, and

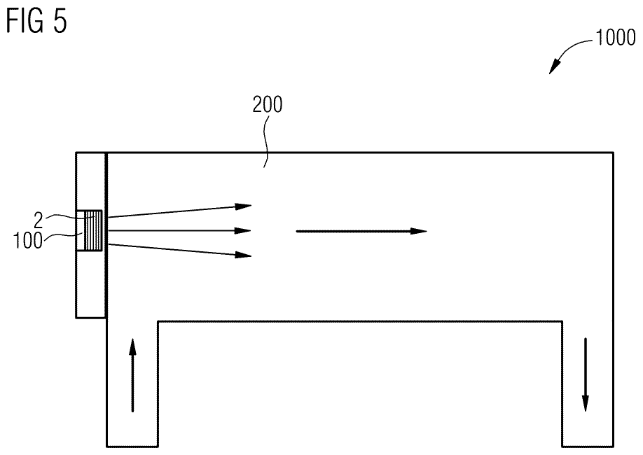

FIG. 5 shows an exemplary embodiment of a disinfection device.

FIG. 1 shows a first exemplary embodiment of an optoelectronic semiconductor chip 100 in a cross-sectional view. The semiconductor chip 100 comprises a semiconductor layer sequence 1 with an active layer 10. The semiconductor layer sequence 1 is based, for example, on AlGaN. The primary radiation generated by the active layer 10 during operation is, for example, radiation with a wavelength of at most 320 nm or at most 280 nm, i.e. UV-B or UV-C radiation.

The semiconductor layer sequence 1 comprises a first side 11 and a second side 13 opposite the first side 11. The semiconductor layer sequence 1 is grown on a growth substrate 4. The growth substrate 4 is a sapphire substrate in the present case. On the side facing the semiconductor layer sequence 1, the sapphire substrate 4 is structured with a structuring 33 in order to deflect the primary radiation upon impingement. The structuring 33 is, for example, a nano-structuring, with structure sizes in the range between 100 nm and 300 nm inclusive. The sapphire substrate 4 can be a so-called nano-PSS.

The structuring 33 is part of a deflection structure 3 for deflecting the primary radiation. The deflection structure 3 also comprises a beveled mesa edge 32 on a side surface 12 of the semiconductor layer sequence 1. The mesa edge 32 extends at an angle of approximately 45° with respect to a main extension plane of the active layer 10. A mirror coating 5, for example a metal layer such as an Ag layer, is applied to the beveled mesa edge 12. The beveled mesa edge 32, like the structuring 33, also serves to redistribute the primary radiation.

Contact elements 51, 52 are applied to the second side 13 of the semiconductor layer sequence 13. The contact elements 51, 52 serve to make electrical contact with the semiconductor layer sequence 1 or the semiconductor chip 100. One of the contact elements is a cathode, the other is an anode. The contact elements 51, 52 can also form a mirror coating 5 on the second side 13 of the semiconductor layer sequence 1.

On a side of the growth substrate 4 opposite the semiconductor layer sequence 1, the growth substrate 4 is also provided with a structuring 33, which is also part of the deflection structure 3 and serves to deflect the primary radiation from the semiconductor layer sequence 1. A planarization layer 6, for example of a material transparent to the primary radiation, is applied to this side of the growth substrate 4. The side of the planarization layer 6 facing away from the growth substrate 4 can be smooth within the manufacturing tolerances, for example with a roughness of at most 5 nm.

An angle-selective filter 2 is applied to the planarized side of the planarization layer 6. The angle-selective filter 2 is a dielectric filter with a plurality of dielectric layers 21, 22 stacked on top of each other. For example, layers with a higher refractive index and layers with a lower refractive index alternate in the dielectric filter 2. Layer 21 is an HfO layer, for example, and layer 22 is an SiO₂ layer, for example. These two layers can be arranged alternately.

The angle-selective filter 2 is configured in such a way that it only let pass primary radiation from the semiconductor layer sequence 1 in a predefined angular range, for example of a maximum of 20° to the main emission direction (normal to the dielectric filter 2). The angular selectivity can be adjusted by the thicknesses of the individual dielectric layers 21, 22. Thicknesses of the dielectric layers are, for example, in the range between λ/8 and λ/2 inclusive, where λ is the wavelength of the primary radiation, in particular the wavelength at which the primary radiation comprises an intensity maximum.

A further mirror coating 5, for example made of a metal, is applied to side surfaces of the growth substrate 4 and side surfaces 12 of the semiconductor layer sequence 1 in order to reflect the primary radiation hitting the side surfaces.

Instead of emitting the primary radiation, the semiconductor chip can also comprise a conversion element that converts the primary radiation of the active layer into UV radiation, which is then subsequently emitted. The angle-selective filter 2 is then configured in particular to filter this UV radiation.

FIG. 2 shows a second exemplary embodiment of the optoelectronic semiconductor chip 100. In contrast to FIG. 1, the growth substrate is removed here. However, the semiconductor layer sequence 1 is stabilized by a housing body 7, which forms a carrier on the second side 13 of the semiconductor layer sequence 1 and forms a frame around the side surfaces 12. The housing body 7 is a plastic body, for example. The semiconductor chip 100 is a so-called chip-size package. The housing body 7 can, for example, be formed from a material that is impermeable to the primary radiation.

In FIG. 2, the first side 11 of the semiconductor layer sequence 1 is structured, for example using an etching process or grinding process. In addition to the beveled mesa edge 32, the structuring or roughening 31 of the first side 11 is part of a deflection structure 3 for deflecting the primary radiation. The roughened first side 11 is covered with a planarization layer 6 and the angle-selective filter 2 is applied directly to the flat/smooth side of the planarization layer 6.

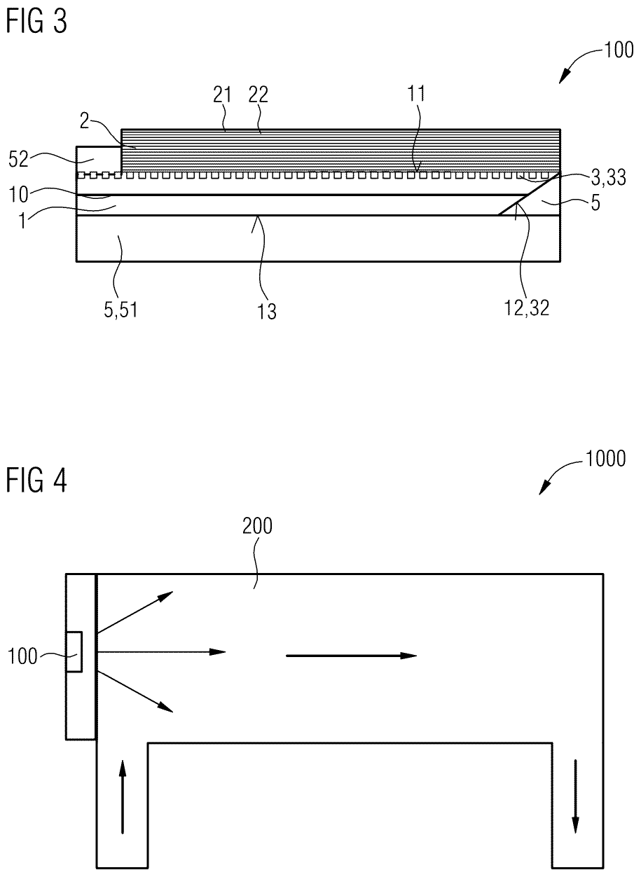

FIG. 3 shows a third exemplary embodiment of the optoelectronic semiconductor chip 100. Unlike in FIG. 2, here not both contact elements 51, 52 are applied to the second side 13, but one of the contact elements 52 is arranged on the first side 11. As a result, unlike in FIG. 2, the angle-selective filter 2 does not cover the entire first side 11 of the semiconductor layer sequence 1, but only between 60% and 90% of the first side 11, for example.

FIG. 4 shows a modification of a disinfection device 1000. The disinfection device 1000 comprises a housing 200 which includes a receiving region through which a fluid, for example a liquid or a gas, can flow. The housing 200 also comprises an inlet and an outlet through which the fluid can be supplied and dissipated. An optoelectronic semiconductor chip 100 is arranged on one side of the housing 200, which emits UV radiation into the receiving region, whereby the fluid can be disinfected.

In FIG. 4, the semiconductor chip 100 emits radiation in a relatively wide angular range so that a relatively large amount of the emitted radiation strikes the housing 200 or the wall of the housing 200 and can be absorbed by the wall of the housing 200. This means that a very high power, for example several semiconductor chips or a larger emission surface, is required to achieve sufficient disinfection.

FIG. 5 shows an exemplary embodiment of the disinfection device 1000 in which a semiconductor chip 100 with an integrated, angle-selective filter 2 is used, for example one of the semiconductor chips 100 of FIGS. 1 to 3. The angle-selective filter 2 allows a much narrower angular range to be achieved than in FIG. 4. As a result, less of the UV radiation emitted by the semiconductor chip 100 is absorbed by the housing 200. For example, almost all of the emitted radiation is radiated into the receiving region or onto the fluid to be disinfected. In this way, a very high degree of efficiency can be achieved.

This patent application claims the priority of the German patent application 10 2021 129 106.2, the disclosure of which is hereby incorporated by reference.

The invention is not limited to the exemplary embodiments by the description thereof. Rather, the invention includes any new feature as well as any combination of features, which includes in particular any combination of features in the patent claims, even if these features or this combination itself is not explicitly stated in the patent claims or exemplary embodiments.

### LIST OF REFERENCE SIGNS

1 semiconductor layer sequence
2 angle-selective filter
3 deflection structure
4 growth substrate
5 mirror coating
6 planarization layer
7 housing body
10 active layer
11 first side
12 side surfaces
13 second side
21 dielectric layer
22 dielectric layer
31 structuring
32 beveled mesa edge
33 structuring
51 contact element
52 contact element
100 optoelectronic semiconductor chip
200 housing
1000 disinfection device

1. Optoelectronic semiconductor chip comprising:
a semiconductor layer sequence with an active layer for generating primary radiation,
an angle-selective filter on a first side of the semiconductor layer sequence, wherein
the semiconductor chip emits radiation in the UV range during operation,
the angle-selective filter is configured to let pass only radiation that hits the filter in a predefined angular range.

2. Optoelectronic semiconductor chip according to claim 1, further comprising:
a growth substrate of the semiconductor layer sequence, wherein
the growth substrate is arranged between the semiconductor layer sequence and the filter.

3. Optoelectronic semiconductor chip according to claim 1, wherein
the filter is a dielectric filter,
the filter comprises a plurality of dielectric layers.

4. Optoelectronic semiconductor chip according to claim 3, wherein
the filter comprises at least one layer comprising HfO and at least one layer comprising SiO₂.

5. Optoelectronic semiconductor chip according to claim 1, further comprising:
a deflection structure for deflecting the radiation generated in the semiconductor chip.

6. Optoelectronic semiconductor chip according to claim 5, wherein
the deflection structure comprises a structuring of the semiconductor layer sequence.

7. Optoelectronic semiconductor chip according to claim 5, wherein
the deflection structure comprises a beveled mesa edge of the semiconductor layer sequence.

8. Optoelectronic semiconductor chip according to claim 1, further comprising:
a mirror coating on one side of the semiconductor layer sequence for reflecting the radiation generated in the semiconductor chip.

9. Optoelectronic semiconductor chip according to claim 1, wherein
the radiation emitted by the semiconductor chip has a maximum intensity at no more than 320 nm.

10. Optoelectronic semiconductor chip according to claim 1, further comprising:
a growth substrate of the semiconductor layer sequence.

11. Optoelectronic semiconductor chip according to claim 5, wherein
the optoelectronic semiconductor chip comprises a growth substrate of the semiconductor layer sequence,
the deflection structure comprises a structuring of one side of the growth substrate.

12. Optoelectronic semiconductor chip according to claim 10, wherein
at least one side surface of the growth substrate is mirror-coated.

13. Optoelectronic semiconductor chip according to claim 1, wherein
the semiconductor chip is a thin-film chip.

14. Optoelectronic semiconductor chip according to claim 1, wherein
the predefined angular range comprises angles of at most 30° with respect to the main emission direction.

15. Disinfection device comprising:
a housing with a receiving region for receiving an object to be disinfected,
an optoelectronic semiconductor chip according to claim 1, wherein
the semiconductor chip is arranged with respect to the housing in such a way that radiation emitted by the semiconductor chip during operation is emitted onto the receiving region.

16. Optoelectronic semiconductor chip comprising:
a semiconductor layer sequence with an active layer for generating primary radiation,
an angle-selective filter on a first side of the semiconductor layer sequence,
a growth substrate of the semiconductor layer sequence, wherein
the semiconductor chip emits radiation in the UV range during operation,
the angle-selective filter is configured to let pass only radiation that hits the filter in a predefined angular range,
the growth substrate is arranged between the semiconductor layer sequence and the filter.

17. Disinfection device comprising:
a housing with a receiving region for receiving an object to be disinfected,
an optoelectronic semiconductor chip comprising
a semiconductor layer sequence with an active layer for generating primary radiation,
an angle-selective filter on a first side of the semiconductor layer sequence,
a growth substrate of the semiconductor layer sequence, wherein
the semiconductor chip emits radiation in the UV range during operation,
the angle-selective filter is configured to let pass only radiation that hits the filter in a predefined angular range,
the growth substrate is arranged between the semiconductor layer sequence and the filter,
the semiconductor chip is arranged with respect to the housing in such a way that radiation emitted by the semiconductor chip during operation is emitted onto the receiving region.
